(19) United States
(12) Patent Application Publication
(10) Pub. No.: US 2025/0120825 A1
(43) Pub. Date: Apr. 17, 2025

(54) IMPLANT HOLDER AND INSERTER DEVICES AND SYSTEMS

(71) Applicant: Medos International Sàrl, Le Locle (CH)

(72) Inventors: Eric Biester, Barrington, RI (US); Michael Sorrenti, Middleboro, MA (US)

(21) Appl. No.: 18/912,692

(22) Filed: Oct. 11, 2024

Related U.S. Application Data
(60) Provisional application No. 63/590,170, filed on Oct. 13, 2023.

(51) Int. Cl. A61F 2/46

(52) U.S. Cl. CPC A61F 2/4611

(57) ABSTRACT

Example instruments disclosed herein can include a bar that can engage a rod slot of a hook or other implant. A user can tighten a setscrew to make a rigid connection between the instrument and the implant. The bar can prevent the need for counter-torque on the implant during engagement and disengagement by virtue of its rigid connection to the implant. The instrument can also include a handle and a shaft extending at an angle from a distal end of the instrument. The handle can be used to both hold and manipulate the implant into place. A mallet can be applied to the handle to transmit insertion forces to the implant to facilitate insertion thereof. Example instruments can include a setscrew captured in a distal housing by one or more pins that allow translation of the setscrew within the distal housing but prevent removal of the setscrew from the housing.

### CROSS-REFERENCE TO RELATED APPLICATIONS

This application claims the benefit of U.S. Provisional Application No. 63/590,170,filed on Oct. 13, 2023. The entire contents of this application are incorporated by reference herein.

### FIELD

This disclosure relates generally to surgical instruments and methods, and more particularly to such instruments and methods that are utilized to manipulate and position implants, such implants utilized in spine or other orthopedic surgery.

### BACKGROUND

Implants can be utilized in a variety of surgical procedures. In some procedures, implants having hook features to attach to patient anatomy are utilized in addition to, or in place of, other types of anchor implants, such as a bone screw, etc. In spine surgery, for example, pedicle hooks can be utilized that press against a portion of a patient's anatomy and provide anchoring and/or stabilization for a spinal fixation rod, tether, or other fixation construct. Pedicle hooks can be utilized in place of pedicle screws in certain cases, and can be combined with screws when building a fixation construct spanning multiple vertebrae. In other embodiments, implants with various hook or hook-like features can be utilized to attach to other implants rather than a portion of patient anatomy. Still further, there are a variety of other implants that may or may not include hook-like features but similarly require instrumentation to grasp and manipulate them during a procedure.

Handling, manipulation, and implantation of hook implants and other types of implants can involve the use of multiple instruments. In the case of a hook implant, for example, these can include a pair of forceps to hold the hook and a pusher rod to engage the hook at an angle and facilitate application of pushing forces that are more in-line with the direction of the hook insertion over a portion of patient anatomy. Further, in some cases use of a mallet can be required to move the implant into a desired position. The mallet can often be applied to the proximal end of the pusher rod such that the pusher rod transfers the mallet force to the implant, and this can require use of three hands to handle the various instruments. This can become cumbersome to a surgeon or other user, lengthen a given procedure involving a hook implant, and increase cost associated with the procedure.

Accordingly, there is a need for improved implant holder and inserter devices and methods to address the shortcomings noted above, among others.

### SUMMARY

Certain aspects of the present disclosure can provide a single instrument that can solve the above-noted drawbacks associated with implant holding and manipulation, e.g., the need for multiple instruments and multiple people to accomplish the task of implant insertion. Certain aspects of the present disclosure can be particularly adapted for use with various types of hook implants.

Example instruments disclosed herein can include a distal bar that can engage a rod slot of a hook or other implant. In operation, a user can tighten an embedded setscrew to make a rigid connection between the bar of the instrument and the implant, something that can be done at a remote location from a surgical site if desired. The distal bar can prevent the need for counter-torque on the implant during engagement and disengagement by virtue of its rigid connection to the implant. The instrument can also include a handle and a shaft rigidly affixed at, for example, a 45-degree angle with a distal end of the instrument. In some examples, the handle can be used to both hold and manipulate the implant into place. Due to the angle of the handle relative to the distal end of the instrument, a mallet can be applied to the handle to transmit insertion forces to the implant in a manner that facilitates insertion of the implant, something that can be particularly advantageous when using an implant having a hook to attach to patient anatomy. Example instruments can include a monolithic embedded setscrew that is captured in a distal housing by one or more pins that allow translation of the embedded setscrew within the distal housing but prevent removal of the setscrew from the housing.

In one aspect, a surgical instrument is disclosed that can include a proximal handle, a shaft extending distally from the proximal handle, and a housing coupled to the distal end of the shaft. The housing can include an upper socket, two opposed arms extending from the upper socket, and a post connecting the two opposed arms at a position farthest from the upper socket. The instrument can further include a setscrew disposed within the housing. The two opposed arms of the housing can define a gap between the post and the upper socket, and the upper socket can include an opening at an end opposite the two opposed arms. The setscrew can be captured within the upper socket and free to translate from a first position, wherein a threaded distal end of the setscrew is disposed within the upper socket, to a second position, wherein the threaded distal end of the setscrew extends into the gap between the two opposed arms.

Any of a variety of alternative or additional features can be included and are considered within the scope of the present disclosure. For example, in some embodiments, the setscrew can be monolithic. Further, in some embodiments the housing can be monolithic.

In certain embodiments, the setscrew can include a proximal end defining a radially outwardly extending flange that is configured to interface with a corresponding shoulder formed in the opening of the upper socket to define a maximum depth of distal insertion of the setscrew. In some embodiments, the housing can include a through-hole formed in a sidewall of the upper socket, and the instrument can further include a pin disposed in the through-hole such that the pin extends into the upper socket to capture the setscrew. In certain embodiments, the housing can include first and second through-holes opposed from one another, and the instrument can further include corresponding first and second pins disposed in the first and second through-holes. In some embodiments, a distance between radially inward most ends of the first and second pins can be less than an outer diameter of the threaded distal end of the setscrew. In certain embodiments, the setscrew can define an intermediate portion arranged longitudinally between the threaded distal end and the proximal end of the setscrew, the intermediate portion having a diameter less than a diameter of the threaded distal end and less than the distance between the radially-inward-most ends of the first and second pins, such that the first and second pins permit translation of the setscrew along a length of the intermediate portion and prevent proximal removal of the setscrew by interfering with the threaded distal end.

In some embodiments, the housing can be coupled with the shaft at one end of the post.

In certain embodiments, a distal-facing surface of the post can be convex.

In some embodiments, the two opposed arms can define a pair of inner sidewalls that are coextensive with an inner surface of the upper socket.

In certain embodiments, the threaded distal end of the setscrew can be sized and shaped to extend laterally beyond the two opposed arms when the threaded distal end extends into the gap between the arms.

In some embodiments, a proximal-facing surface of the setscrew can be configured to be flush with a proximal end of the upper socket when the setscrew is in the second position.

In certain embodiments, a longitudinal axis of the post and a longitudinal axis of the shaft can be coplanar.

In some embodiments, a longitudinal axis of the post and a longitudinal axis of the shaft can be obliquely angled relative to one another in a plane that is perpendicular to a longitudinal axis of the upper socket.

In certain embodiments, the setscrew can be biased toward the opening of the upper socket opposite the two opposed arms.

In another aspect, a surgical instrument is disclosed that can include a proximal handle, a shaft extending distally from the proximal handle, and a housing coupled to the distal end of the shaft. The housing can include an upper socket with a through-hole formed in a sidewall thereof, an arm extending from the upper socket, and a post extending from the arm at a position farthest from the upper socket. The instrument can further include a setscrew disposed within the housing and a pin disposed in the through-hole such that the pin extends into the upper socket to capture the setscrew.

As with the instrument described above, any of a variety of alternative or additional features can be included and are considered within the scope of the present disclosure. For example, in some embodiments, the upper socket can include first and second through-holes opposed from one another, the instrument further comprising corresponding first and second pins disposed in the first and second through-holes. In certain embodiments, a distance between radially inward most ends of the first and second pins can be less than an outer diameter of a threaded distal end of the setscrew. In some embodiments, the setscrew can define an intermediate portion arranged longitudinally between the threaded distal end and a proximal end of the setscrew, the intermediate portion having a diameter less than a diameter of the threaded distal end and less than the distance between the radially-inward-most ends of the first and second pins, such that the first and second pins permit translation of the setscrew along a length of the intermediate portion and prevent proximal removal of the setscrew by interfering with the threaded distal end.

In certain embodiments, the setscrew can include a proximal end defining a radially outwardly extending flange that is configured to interface with a corresponding shoulder formed in an opening of the upper socket at an end opposite the arm to define a maximum depth of distal insertion of the setscrew.

In some embodiments, the setscrew can be biased toward an end of the upper socket that is opposite the arm extending from the upper socket.

In another aspect, a method of assembling an instrument is disclosed that can include inserting a setscrew into a proximal-facing opening of an upper socket of a housing, the setscrew including a threaded distal end, a proximal end defining a radially-outward extending flange, and an intermediate portion extending therebetween that has a diameter less than the threaded distal end and the radially-outward extending flange. The method can further include inserting first and second pins into opposed first and second through-holes formed in a sidewall of the upper socket such that a distance between radially-inward-most ends of the first and second pins is less than an outer diameter of the threaded distal end of the setscrew, less than a diameter of the radially-outward extending flange of the setscrew, and greater than a diameter of the intermediate portion of the setscrew such that the first and second pins capture the setscrew within the upper socket while allowing translation of the setscrew relative to the upper socket along a length of the intermediate portion of the setscrew.

As with the instruments described above, the methods disclosed herein can include any of a variety of additional or alternative steps that are considered within the scope of the present disclosure. For example, in some embodiments, the method can further include coupling a shaft to the housing. In certain embodiments, the method can further include coupling a handle to the shaft.

In some embodiments, the method can further include extending a post of the housing that is positioned distal to the upper socket into a recess of an implant. In certain embodiments, the method can further include distally extending the setscrew relative to the upper socket such that the threaded distal interfaces with threads formed in the recess of the implant. In some embodiments, the method can further include rotating the setscrew relative to the upper socket to urge a distal-facing surface of the upper socket into contact with a proximal-facing surface of the implant and create a rigid coupling between the housing and the implant.

Any of the features or variations described herein can be applied to any particular aspect or embodiment of the present disclosure in a number of different combinations. The absence of explicit recitation of any particular combination is due solely to avoiding unnecessary length or repetition.

### BRIEF DESCRIPTION OF THE DRAWINGS

The aspects and embodiments of the present disclosure can be more fully understood from the following detailed description taken in conjunction with the accompanying drawings, in which:

FIG. 1 is a perspective view of one embodiment of an implant holder and inserter device example of the present disclosure;

FIG. 2A is a side view of the device of FIG. 1;

FIG. 2B is side detail view of a distal portion of the device of FIG. 1;

FIG. 2C is a front view of the device of FIG. 1;

FIG. 2D is a cross-sectional view of a distal end portion of the device of FIG. 1 taken through plane B-B of FIG. 2C;

FIG. 3A is a top view of the distal end portion of the device of FIG. 1;

FIG. 3B is a cross-sectional view of the distal end portion of the device of FIG. 1 taken through plane A-A of FIG. 3A;

FIG. 3C is a detail view of the circled portion of FIG. 3B;

FIG. 3D is a front view of the distal end portion of the device of FIG. 1;

FIG. 3E is a cross-sectional view of the distal end portion of the device of FIG. 1 taken through plane B-B of FIG. 3B;

FIG. 3F is a detail view of the circled portion of FIG. 3E;

FIG. 4A is a side view of the setscrew of the distal end portion of the device of FIG. 1;

FIG. 4B is a cross-sectional view of the setscrew of FIG. 4A taken through plane A-A of FIG. 4A;

FIG. 5A is a side view of the shaft of the device of FIG. 1;

FIG. 5B is a cross-sectional view of the shaft of FIG. 5A taken through plane A-A of FIG. 5A;

FIG. 5C is a cross-sectional view of the shaft of FIG. 5A taken through plane B-B of FIG. 5A;

FIG. 5D is a detail view of the circled portion of FIG. 5B;

FIG. 6A is a perspective view of the device of FIG. 1 in proximity to one embodiment of an implant;

FIG. 6B is a cross-sectional view of the device and implant of FIG. 6A taken through plane A-A of FIG. 6E;

FIG. 6C is a cross-sectional view of the device and implant of FIG. 6A taken through the plane A-A of FIG. 6E and showing the distal end portion of the device positioned within a rod-receiving portion of the implant;

FIG. 6D is a cross-sectional view of the device and implant of FIG. 6A taken through the plane A-A of FIG. 6E and showing with the distal end portion of the device positioned within a rod-receiving portion of the implant and a setscrew threaded into the rod-receiving portion of the implant to create a rigid connection between the device and implant;

FIG. 6E is a perspective view of the device and implant of FIG. 6A in the configuration of FIG. 6D;

FIG. 6F is a cross-sectional view of the device and implant of FIG. 6A taken through plane B-B of FIG. 6E in the configuration of FIG. 6D;

FIG. 7A is a perspective view of the device of FIG. 1 in proximity to an alternative embodiment of an implant;

FIG. 7B is a cross-sectional view of the device and implant of FIG. 7A taken through the plane A-A of FIG. 7C;

FIG. 7C is a perspective view of the device and implant of FIG. 7A showing the distal end portion of the device positioned within a rod-receiving portion of the implant to create a rigid connection between the device and implant; and

FIG. 7D is a cross-sectional view of the device and implant of FIG. 7A taken through the plane B-B of FIG. 7C in the configuration of FIG. 7C.

### DETAILED DESCRIPTION

Certain example embodiments will now be described to provide an overall understanding of the principles of the structure, function, manufacture, and use of the devices, systems, and methods disclosed herein. One or more examples of these embodiments are illustrated in the accompanying drawings. The devices, systems, and methods specifically described herein and illustrated in the accompanying drawings are non-limiting embodiments. The features illustrated or described in connection with one embodiment may be combined with the features of other embodiments. Such modifications and variations are intended to be included within the scope of the present disclosure.

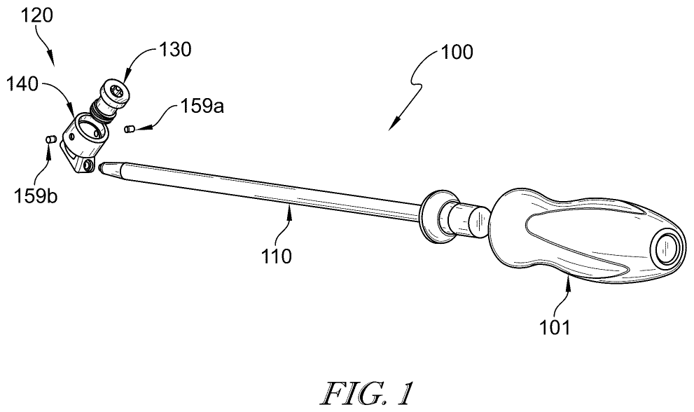

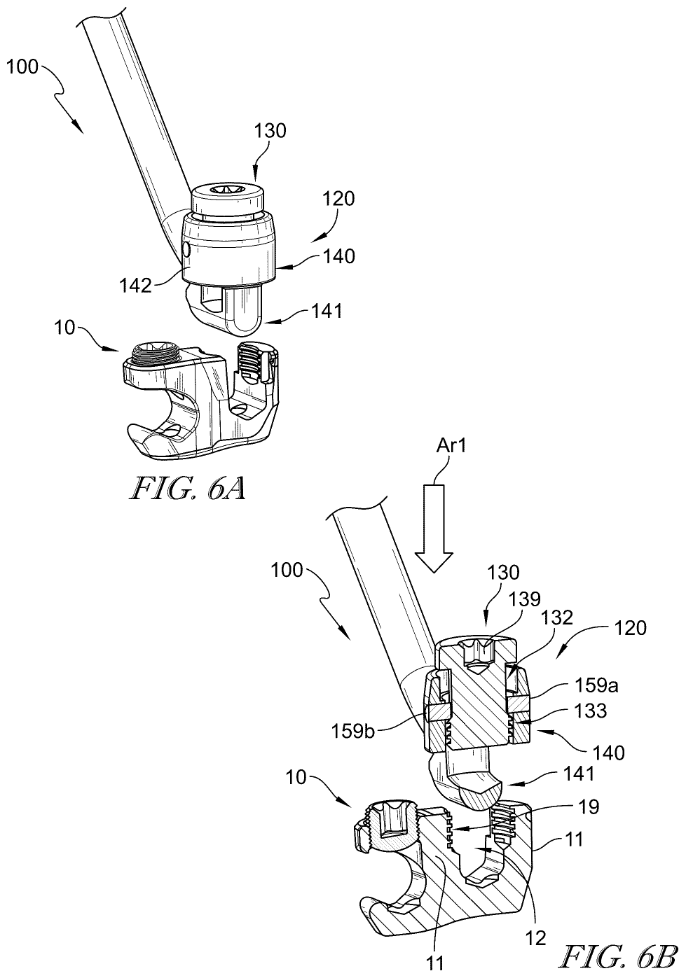

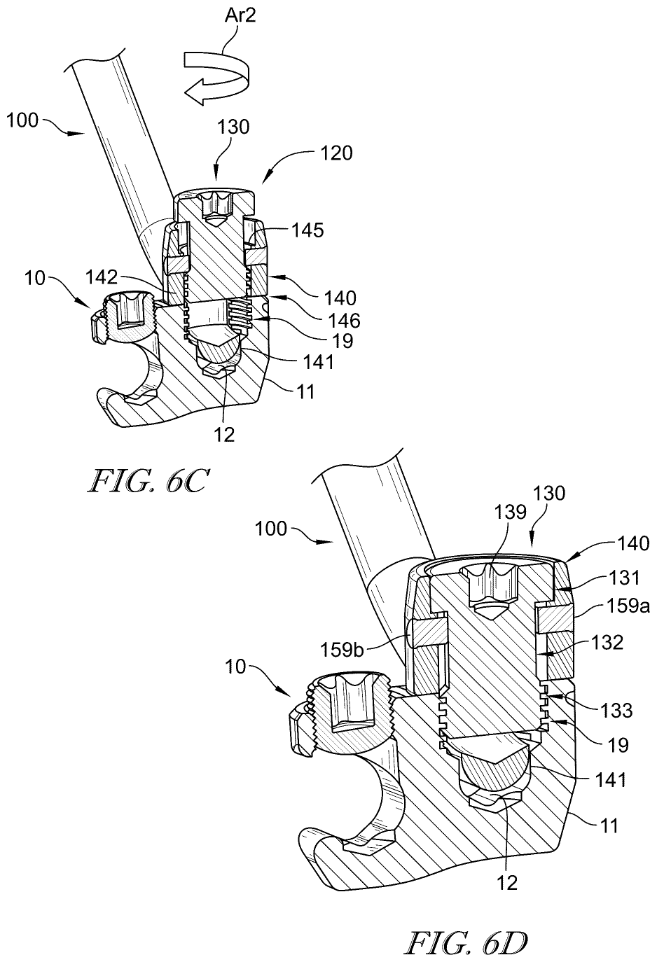

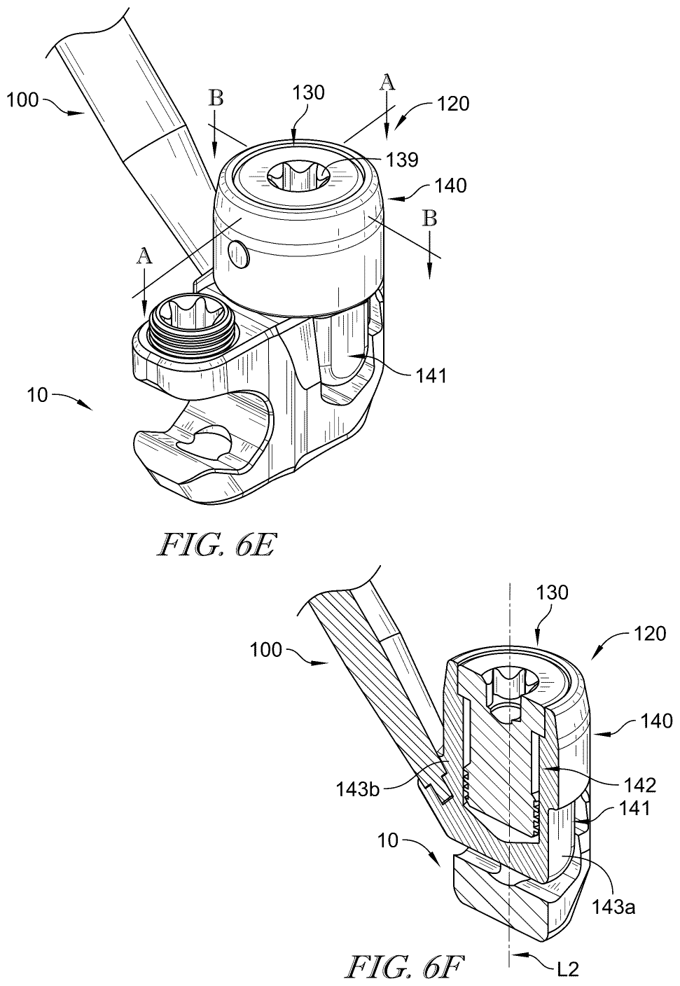

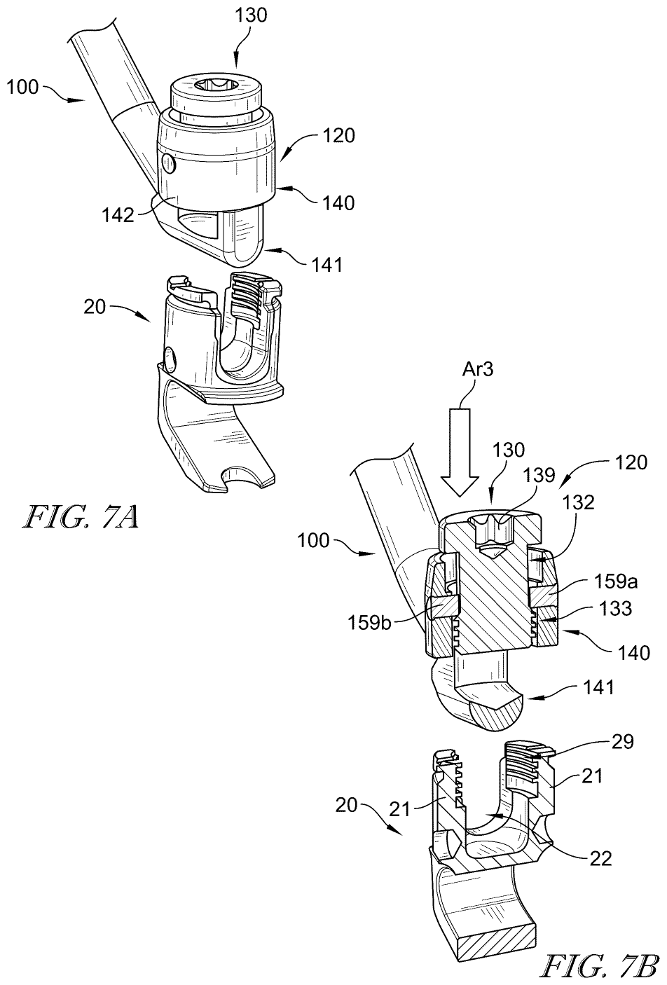

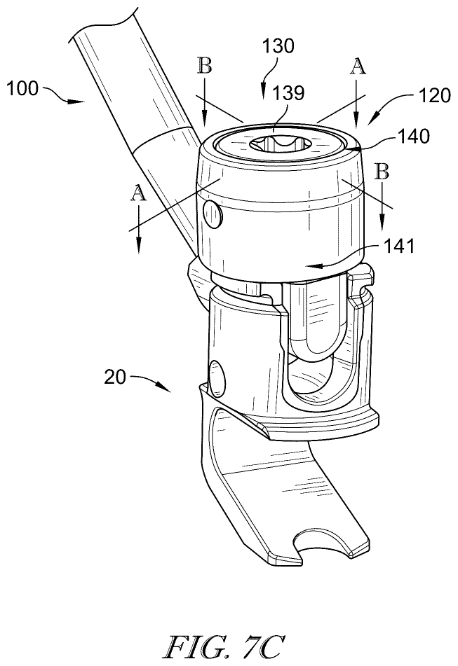

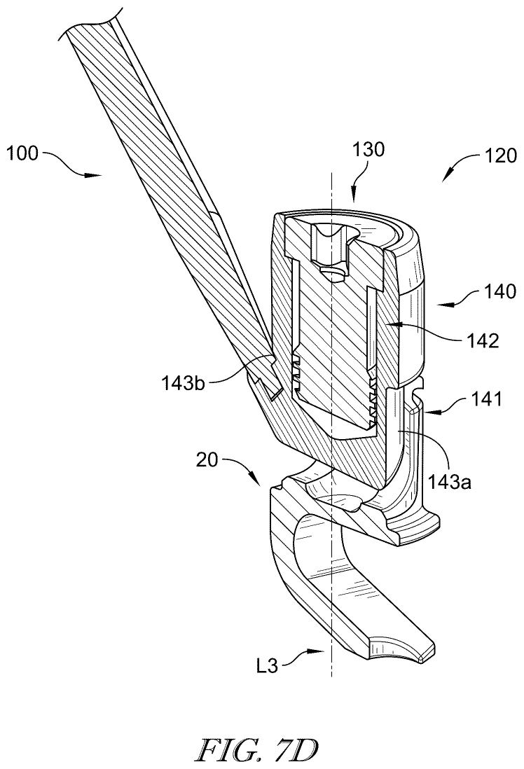

FIGS. 1-5D show one embodiment of an implant holder and inserter device 100, FIGS. 6A-6F illustrate the device 100 in use with an example implant, a spinal rod connector 10, and FIGS. 7A-7D illustrate the device 100 in use with another example implant, a pedicle hook 20. FIGS. 6A-6F and 7A-7D show the coupling of the distal end portion of the device with a rod-receiving recess of the implant such that, once coupled, the two components are rigidly mated and the holder and inserter device can manipulate the implant securely. The distal end portion of the holder and inserter device can include an embedded monolithic setscrew for coupling with the implant, the setscrew being captured in the distal end portion of the device, as explained in more detail below.

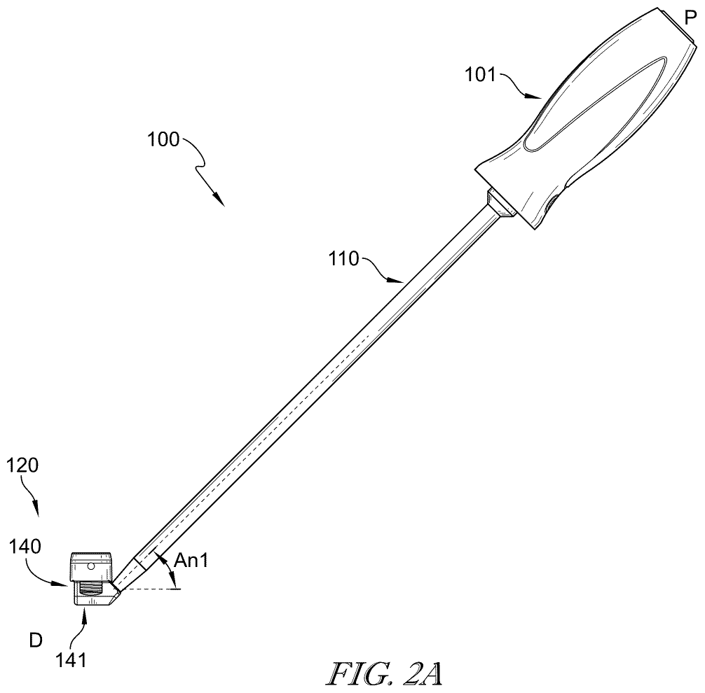

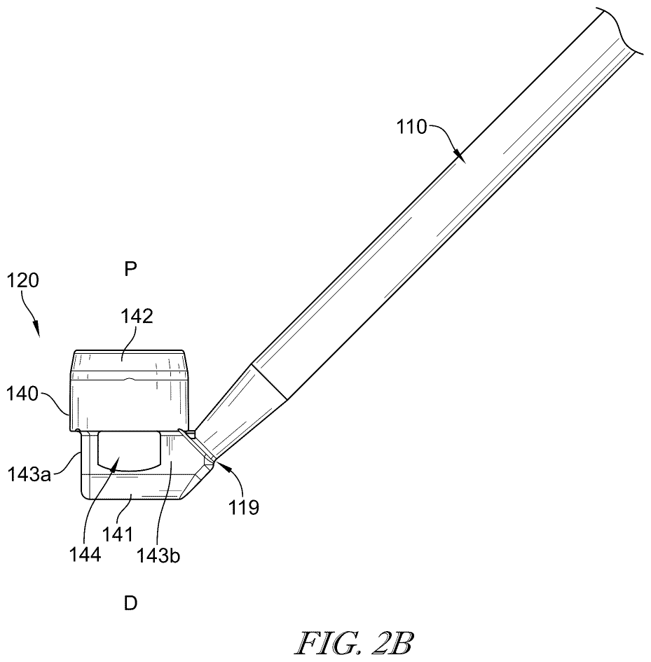

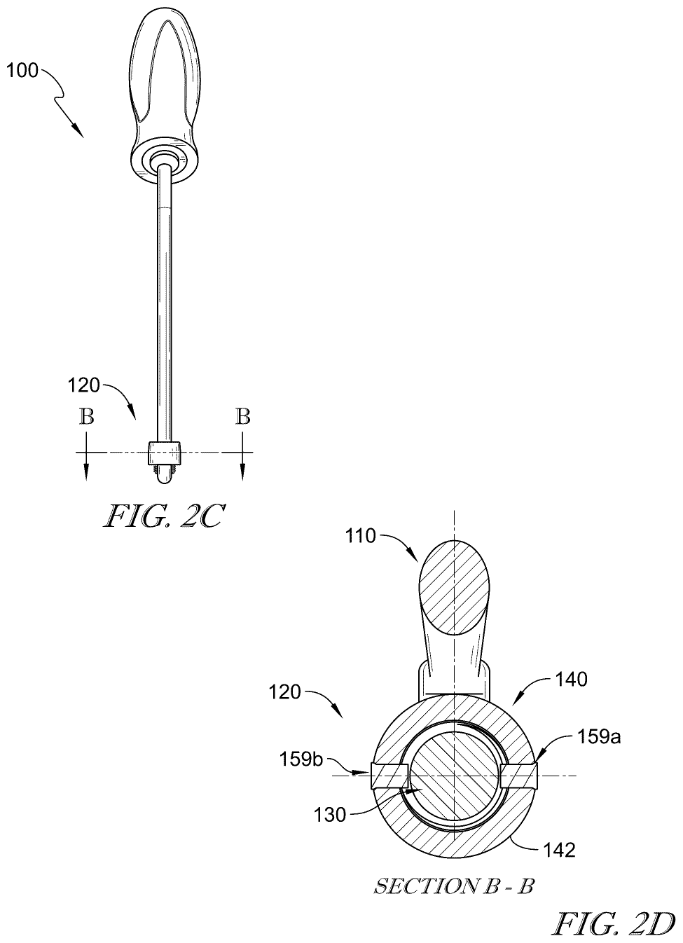

More particularly, FIG. 1 is a perspective view of one embodiment of an implant holder and inserter device 100 (also referred to herein, interchangeably, as an instrument or a hook holder and inserter device). The device 100 can include a proximal handle 101 that can be connected to a distal end portion 120 by an elongated shaft 110. The distal end portion 120 can include a housing 140, a setscrew 130, and pins 159a, 159b that can be arranged to capture the setscrew 130 into the housing 140. The housing 140, as shown in more detail below, can include a recess for receiving a distal end of the elongated shaft 110 such that, when coupled, the proximal handle 101 can enable manipulation of the housing 140. FIG. 2A is a side view of the implant holder and inserter device 100 of FIG. 1, showing a post 141 that is arranged at a distal end of the housing 140 and is sized and shaped to engage with a surgical implant via, for example, a rod-receiving recess of the implant. In some embodiments, the post 141 can have a distal-facing surface that defines a radius similar to, or the same as, that of a rod configured to be secured to the implant that is being coupled to the inserter device 100. FIG. 2A also shows that the shaft 110 and handle 101 extend at an angle An1 relative to the post 141. In some embodiments, this angle can be about 45 degrees, though in other embodiments other angles can be utilized. The angled extension of the handle 101 and shaft 110 relative to the post 141 and any implant coupled to the distal portion 120 of the device 100, can allow advantageous transfer of impact forces by a mallet or other instrument from the proximal handle 101 to the implant. This can be advantageous when coupling the device 100 to a pedicle hook or similar implant and attempting to position it over patient anatomy.

FIG. 2B shows additional details of the distal end portion 120. The housing 140 can be connected to a distal end 119 of the elongated shaft 110 and can include an upper socket 142 for holding the setscrew 130. The post 141 can be connected to the socket 141 by two arms 143a, 143b that can be spaced apart to define a gap 144 between the arms 143a, 143b that allows the setscrew to protrude therefrom. Movement of the setscrew 130 and its protrusion through the gap can permit threaded engagement of the distal end of the setscrew 130 with an implant, e.g., a rod-receiving recess of the implant in which the post 141 of the housing 140 is disposed.

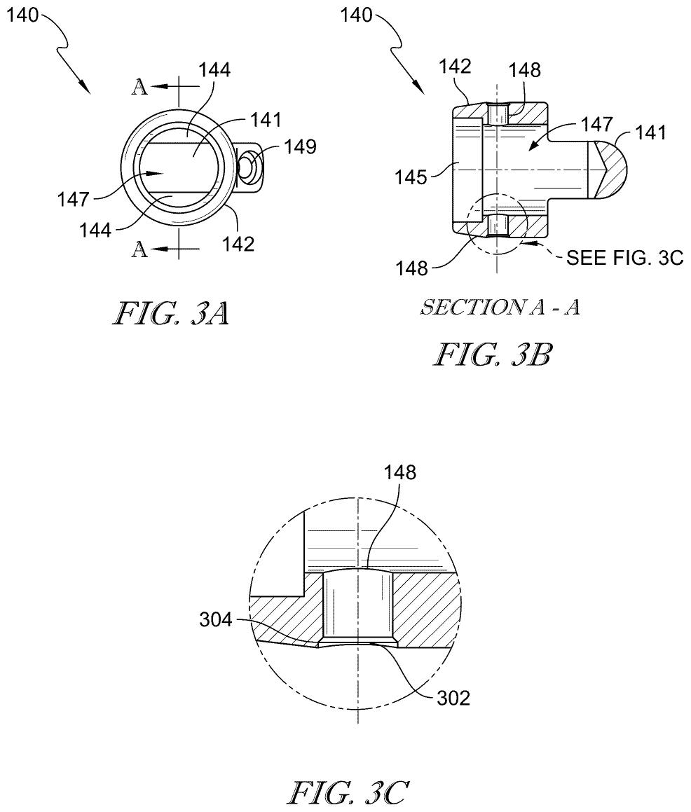

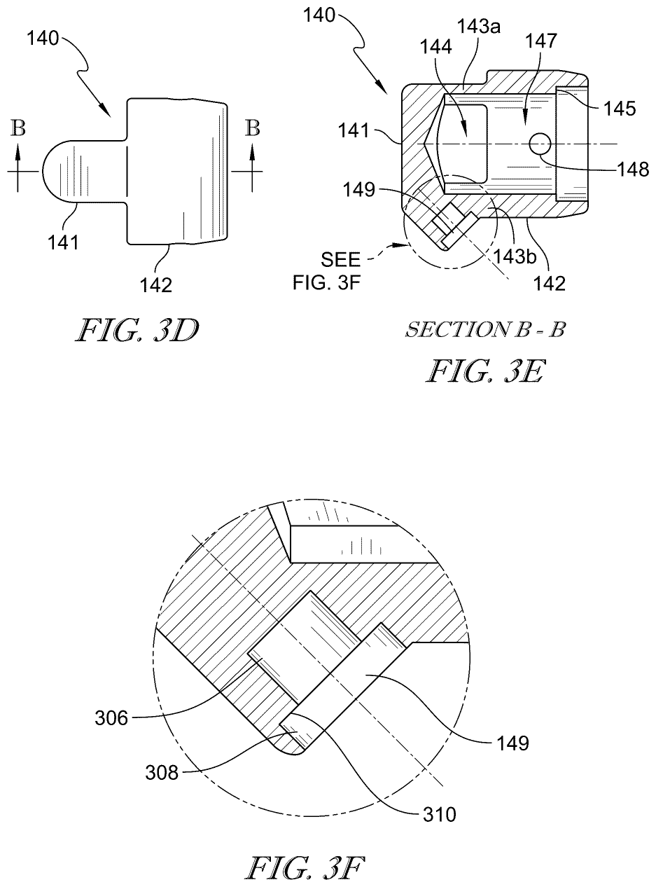

FIG. 2C shows a front-view of the inserter device 100 and FIG. 2D is a cross-sectional view through plane B-B of FIG. 2C. FIG. 2D shows the setscrew 130 disposed in the upper socket 142 with opposing pins 159a, 159b extending into the upper socket 142. In operation, the pins 159a, 159b effectively capture the setscrew 130 in the upper socket 142 by preventing removal of the setscrew 130 in a proximal direction (i.e., out of the page in the view of FIG. 2D) by interfering with the distal threads of the setscrew (i.e., the distal threads define a radius larger than the distance between the inner ends of the pins 159a, 159b). Additionally, either or both of the pins 159a, 159b and the upper socket 142 itself can limit distal advancement of the setscrew. For example, a proximal end of the setscrew 130 can define a radially-outwardly extending flange, which can be configured to abut the pins 159a, 159b and/or a shoulder formed in the upper socket 142, as shown in more detail in FIGS. 3E, 6C, and 6D. The pins 159a, 159b can enable a monolithic setscrew to be captured in the upper socket 142 and, in some embodiments, the pins 159a, 159b can be removable and/or moveable such that the setscrew 130 can be selectively captured in, and removed from, the upper socket 142.

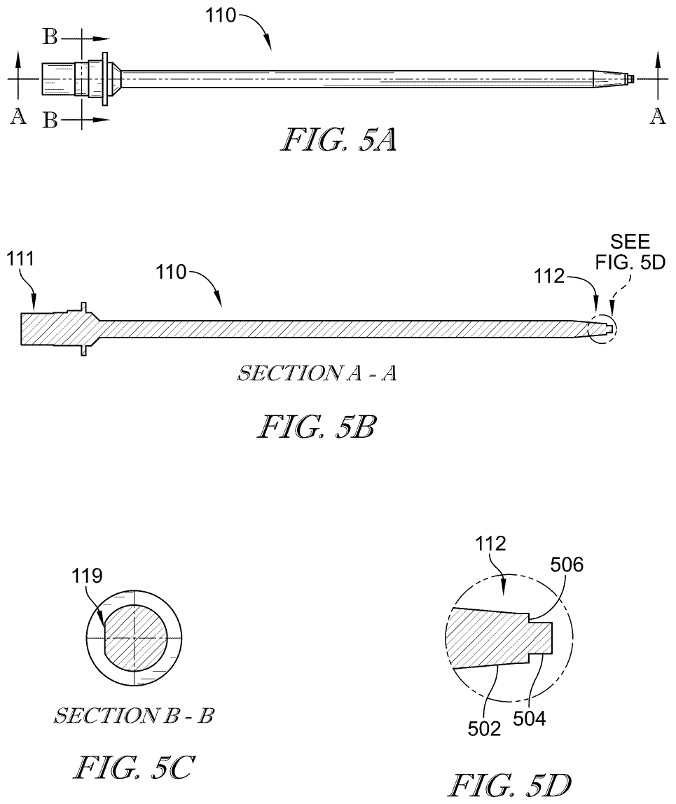

FIGS. 3A-3F show additional details of the housing 140 without the setscrew 130 disposed in the upper socket 142. FIG. 3A is a top-down view of the housing 140 and shows that the upper socket 142 can be generally cylindrical with an open view distally of the post 141 that extends across a distal bottom portion of the housing 140, as well as the gaps 144 on each side of the post 141. The housing 140 can also include a recess 149 configured to receive and couple with a distal end of the elongated shaft 110 (not shown, see FIGS. 5A-5D). FIG. 3B is a cross-sectional view through plane A-A of FIG. 3A and shows that the upper socket 142 can have sidewalls with opposing holes 148 for receiving pins 159a, 159b (not shown, see FIG. 1), as well as a shoulder 145 formed in a proximal side of the housing for limiting the distal travel of the setscrew 130 (not shown, see FIG. 1). The post 141 can have a distal-facing surface that is radiused and can be, for example, sized and shaped similar to a spinal rod, such that the post 141 can be disposed in a rod-receiving receiving of a spinal implant. Additionally, the post 141 can be disposed distally below the lowest extent of the upper socket 142, such at the post 141 can be disposed in a rod-receiving recess and the threads of the setscrew 130 (not shown, see FIG. 1) disposed in the upper socket 142 can extend distally towards the post 141 and engage with corresponding threads on an inner surface of the implant rod-receiving recess. FIG. 3C shows additional details of one of the holes 148 for the pins 159a, 159b (not shown, see FIG. 1). These can include, for example, a beveled exterior rim 302 to enable a flush insertion of a pin with a lip 304 at a proximal end to prevent over insertion.

FIG. 3D is a front view of the housing 140 and FIG. 3E is a cross-sectional view through plane B-B of FIG. 3D, which cuts through the arms 143a, 143b to show that the arms 143a, 143b define an inner surface with a same diameter as the upper socket 142, thus allowing free movement of the setscrew 130 (not shown, see FIG. 1) distal to the upper socket 142 (i.e., enabling the shoulder 145 to limit the insertion depth of the setscrew 130 within a cavity 147 defined by the interior walls of the upper socket 142. Additionally, FIG. 3F shows details of the recess 149 configured to receive and couple with a distal end of the elongated shaft 110 (not shown, see FIGS. 5A-5D). In particular, the recess 149 can be threaded such that it can receive a threaded distal end of the elongated shaft 110 and thereby facilitate rigid coupling between the housing 140 and the shaft 110. In addition, the recess 149 can include multiple stepped portions 306, 308 having different diameters that can facilitate more robust coupling between the housing 140 and the shaft 110. For example, a distal portion 306 of the recess 149 can be threaded while a proximal portion 306 can be smooth, thereby forming a shelf 310 that can abut a shoulder formed on the shaft 110.

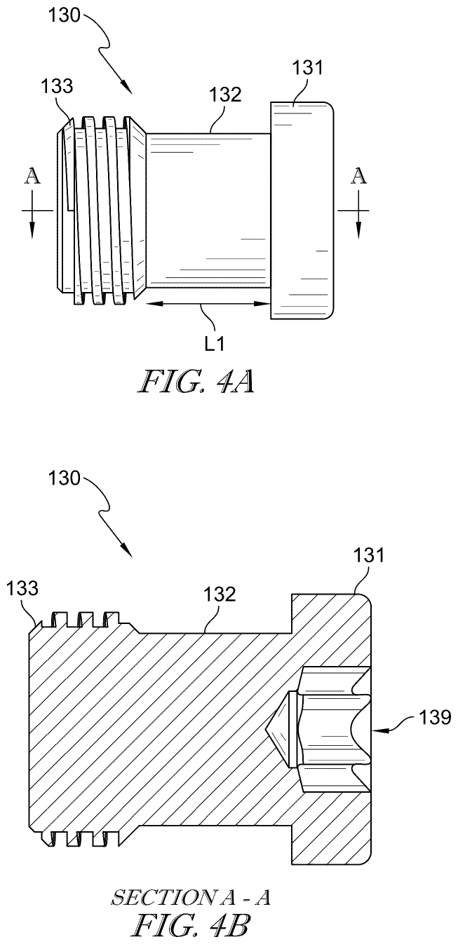

FIG. 4A is a side view of the setscrew 130 and FIG. 4B show a cross-sectional view through plane A-A of FIG. 4A. The setscrew 130 can be constructed from one or more individual components or can have a monolithic design. The setscrew 130 can include distal threads 133, a proximal socket or drive feature 139 for receiving a driver, and a proximal flange 131. The setscrew can allow for a strong and reliable connection between the housing 140 and a surgical implant to which the housing 140 is coupled (e.g., via threaded engagement with the threads 133 of the setscrew 130). In addition, the setscrew 130 can include a smooth intermediate portion 132 disposed between the proximal flange 131 and the distal threads 133. The intermediate portion 132 can have an axial height L1 spanning from the distal end of the flange 131 to the proximal end of the threads 133. This height L1 can represent the amount of axial translation permitted between the setscrew 130 and the housing 140 when the setscrew is captured within the housing by the pins 159a, 159b because the pins can ride along the intermediate portion 132 without interference, but can interfere with the proximal flange 131 or the distal threads 133.

FIGS. 5A-5D show additional features of the elongated shaft 110 for coupling the handle 101 to the distal assembly 120. FIGS. 5A and 5B show side views of the elongated shaft 110, which can include a proximal portion 111 configured to accept the proximal handle 101 (not shown, see FIG. 1) and a distal portion 112 configured to be inserted into the recess 149 of the housing 140. FIG. 5C shows that the proximal portion 111 of the elongated shaft 110 can have a generally cylindrical shape with an asymmetric feature 119, such as a flat, etc., to assist in alignment and coupling of the proximal handle 101, as well as torque transfer from the handle through the shaft 110. Additionally, FIG. 5D shows details of the distal portion 112 of the elongated shaft 110, including one or more stepped portions of different diameters, where threads can be included on one or more such portions and shoulders created by step transitions can facilitate robust coupling with the housing 140. For example, the distal portion 112 can include a first portion 502 with a distally tapering diameter and a step transition to a second portion 504 that includes threads formed on an outer surface thereof. Referring back to FIG. 3F, the threaded second portion 504 can interface with the threaded portion 306 of the recess 149 and the first portion 502 can be received within the smooth portion 308 of the recess 149 such that a shoulder 506 on the shaft 110 can abut against the shoulder 310 of the recess 149.

FIGS. 6A-6F show the steps of coupling the device 100 with an implant, such as the spinal rod connector 10. Coupling the device 100 to the connector 10 can include disposing the post 141 of the housing 140 of the distal portion 120 in a rod-receiving recess 12 of the connector 10 and subsequently threading the setscrew 130 captured in the upper socket 142 of the housing 140 into corresponding threads 19 in the rod-receiving recess 12.

While FIGS. 6A-6F illustrate coupling the device 100 with a spinal rod connector 10, any number of different implants and hardware can be coupled with the device 100 via the distal end portion 120. Any such implant or hardware can include an area to receive the post 141 and threads to interface with the setscrew 130, such that tightening the setscrew affixes the implant or hardware to the distal end portion 120. As a result, the device 100 can be utilized with any of a variety of implants that include, e.g., features similar to the connector 10, such as the U-shaped rod-receiving recess with threaded inner surfaces. These can include pedicle hooks (as shown in FIGS. 7A-7D), other types of spinal rod connectors, etc.

FIGS. 6A and 6B show the distal end portion 120 of the device 100 positioned above and proximate to, though separated from, a connector 10. The connector 10 can include a rod-receiving recess 12 defined by two opposing arms 11. An upper, inward-facing portion of the arms 11 can define a threaded portion 19 that can interface with the threads 133 of the setscrew 130. Before interfacing with the threads 19, however, the setscrew 130 can be free to translate within the upper socket 142 over the length of the intermediate portion 132 (see FIG. 4B) and is shown in FIGS. 6A and 6B in a fully withdrawn (i.e., proximally urged) direction, such that the pins 159a, 159b abut a proximal end of the threads 133 of the setscrew 130 to prevent further proximal movement between the setscrew 130 and the housing 140. To couple the distal portion 120 of the device 100 to the connector 10, the device 100 can be moved in the direction of arrow Ar1 in FIG. 6B such that the post 141 passes into the rod-receiving recess 12 of the connector 10, as shown in the configuration of FIG. 6C. In such a configuration, a distal end (e.g., a border surrounding distal opening 146) of the upper socket 142 can abut upper, proximal-facing surfaces of the arms 11 that define the proximal opening to the rod-receiving recess 12. Alternatively, or additionally, the position of the housing 140 with respect to the connector 10 can be defined by the post 141 contacting the connector 10, and/or any other contact between the connector 10 and the distal portion 120.

From the position of the distal portion 120 of the device 100 and the connector 10 in FIG. 6C, the setscrew 130 can be rotated to interface its threads 133 with the threads 19 of the connector 10. For example, the setscrew 130 can be rotated clockwise in the direction of arrow Ar2 in FIG. 6C until the setscrew is in the configuration of FIG. 6D. FIG. 6F shows that the arms 143a, 143b of the housing 140 can define inner walls that can be coextensive with the interior walls of the cavity 147 of upper socket 142, such that the setscrew 130 can freely translate along the longitudinal axis L2 of the cavity during engagement with the threads 19 of the rod-receiving recess 12 of the connector 10. Additionally, and as shown, the post 141 can be configured to be recessed within the rod-receiving recess 12, such that the post 141 does not extend beyond the connector 10 when coupled thereto.

In FIGS. 6D-6F, the distal threaded advancement of the setscrew 130 (e.g., by a driver tip applying torque to the setscrew via the proximal socket or drive feature 139) can dispose the proximal surface of the setscrew 130 flush with the proximal end of the upper socket 142 and can dispose the threaded portion 133 of the setscrew within the gap 144 between the arms 143a, 143b of the housing 140. In addition, the proximal flange 131 of the setscrew 130 can abut the shoulder 145 of the upper socket 142, such that the tightened setscrew 130 can securely affix the connector 10 to the device 100 via the housing 140 and setscrew.

With the distal portion 120 of the device 100 coupled to the connector 10, the connector 10 can be manipulated by the device 100, such as by a user engaging with the proximal handle 101 to move and/or apply a force to the connector 10. Given the rigid coupling between the device 100 and the connector 10, a variety of forces can be transferred to the connector or other implant through the device 100, including counter-torque forces, impact forces for implanting (e.g., applying a mallet to a proximal portion of the device 100), etc. In addition, methods of utilizing the device 100 can include coupling the device 100 to an implant at a location remote from a surgical site, e.g., a surgical technician or other user could couple the device to an implant at a “back table” or other staging area, then pass the assembled implant and device to a surgeon or other user to position the implant at a surgical site. Still further, coupling and de-coupling the device 100 to an implant can be done without application of counter-torque forces to the implant because the post 141 prevents relative rotation between the implant and the housing 140 of the device 100 during application of torque to the setscrew 130.

While the post 141 of the housing 140 can extend in a manner co-planar with the elongated shaft 110, as shown in the illustrated embodiments, other arrangements are also possible. For example, the post 141 can be perpendicular or otherwise obliquely angled relative to the elongated shaft 110 to define a different angle between a connector or other implant coupled to the device 100 and the proximal handle 101. Additionally, the various components of the device, including the housing 140, setscrew 130, shaft 110, among others, can be monolithic in some embodiments or can be constructed as an assembly of one or more individual pieces. Likewise, various components of the device shown as separate pieces here, including the housing 140, handle 101, and shaft 110, among others, can be formed as monolithic components. For example, the housing 140 and the elongated shaft 101 can be a single monolithic component in some embodiments.

Additionally, while the setscrew 130 is shown captured within the upper socket 142 of the housing 140 by pins 159a, 159b, other arrangements are possible, such as a sleeve, snap ring, or other insert that can be placed within the upper socket and can interface with an inner wall of the socket to capture the setscrew against proximal withdrawal from the socket. Moreover, the setscrew 130 in the illustrated embodiments translates freely (as much as permitted by the pins 159a, 159b) relative to the housing 140. In some embodiments, however, the setscrew 130 can be biased in one direction by, for example, a wave washer or other component disposed between the setscrew 130 and housing 140. For example, in certain embodiments the setscrew 130 can be biased upward or proximally relative to the housing 140, i.e., into the configuration shown in FIGS. 6A and 6B. This configuration can facilitate easier coupling of the device 100 to an implant because it maintains the distal portion of the setscrew 130 within the socket 142 and prevents possible interference between a proximal end of the implant and a distal end of the setscrew 130. By way of further explanation, if the setscrew 130 were to be in the distal-most position shown in FIG. 6D prior to coupling with an implant as shown in that figure, its distal end protruding from the gap 144 can interfere with insertion of the post 141 into the recess of the implant. Biasing the setscrew 130 proximally can help prevent such interference.

FIGS. 7A-7D show the steps of coupling the device 100 with another embodiment of an implant, a pedicle hook 20. Coupling the device 100 to the hook implant 20 can include disposing the post 141 of the housing 140 of the distal portion 120 in a rod-receiving recess 22 of the implant 20 and subsequently threading the setscrew 130 captured in the upper socket 142 of the housing 140 into corresponding threads 29 in the rod-receiving recess 22.

FIGS. 7A and 7B show the distal end portion 120 of the device 100 positioned above and proximate to, though separated from, the hook implant 20. The implant 20 can include a rod-receiving recess 22 defined by two opposing arms 21. An upper, inward-facing portion of the arms 21 can define a threaded portion 29 that can interface with the threads 133 of the setscrew 130. Before interfacing with the threads 29, however, the setscrew 130 can be free to translate within the upper socket 142 over the length of the intermediate portion 132 (see FIG. 4B) and is shown in FIGS. 7A and 7B in a fully withdrawn (i.e., proximally urged) direction, such that the pins 159a, 159b abut a proximal end of the threads 133 of the setscrew 130 to prevent further proximal movement between the setscrew 130 and the housing 140. To couple the distal portion 120 of the device 100 to the implant 20, the device 100 can be moved in the direction of arrow Ar3 in FIG. 7B such that the post 141 passes into the rod-receiving recess 22 of the implant 20, as shown in the configuration of FIG. 7C. In such a configuration, a distal end (e.g., a border surrounding distal opening 146) of the upper socket 142 can abut upper, proximal-facing surfaces of the arms 21 that define the proximal opening to the rod-receiving recess 22. Alternatively, or additionally, the position of the housing 140 with respect to the implant 20 can be defined by the post 141 contacting the implant 20, and/or any other contact between the implant 20 and the distal portion 120.

Once the device 100 is inserted into the rod-receiving recess 22 of the implant 20, the setscrew 130 can be rotated to interface its threads 133 with the threads 29 of the implant 20. For example, the setscrew 130 can be rotated clockwise until the setscrew is in the configuration of FIG. 7C. FIG. 7D shows that the arms 143a, 143b of the housing 140 can define inner walls that can be coextensive with the interior walls of the cavity 147 of upper socket 142, such that the setscrew 130 can freely translate along the longitudinal axis L3 of the cavity during engagement with the threads 29 of the rod-receiving recess 22 of the hook implant 20. Additionally, and as shown, the post 141 can be configured to be recessed within the rod-receiving recess 22, such that the post 141 does not extend beyond the implant 20 when coupled thereto.

In FIGS. 7C and 7D, the distal threaded advancement of the setscrew 130 (e.g., by a driver tip applying torque to the setscrew via the proximal socket or drive feature 139) can dispose the proximal surface of the setscrew 130 flush with the proximal end of the upper socket 142 and can dispose the threaded portion 133 of the setscrew within the gap 144 between the arms 143a, 143b of the housing 140. In addition, the proximal flange 131 of the setscrew 130 can abut the shoulder 145 of the upper socket 142, such that the tightened setscrew 130 can securely affix the hook implant 20 to the device 100 via the housing 140 and setscrew.

With the distal portion 120 of the device 100 coupled to the hook implant 20, the implant can be manipulated by the device 100, such as by a user engaging with the proximal handle 101 to move and/or apply a force to the implant 20. Given the rigid coupling between the device 100 and the implant 20, a variety of forces can be transferred to the connector or other implant through the device 100, including counter-torque forces, impact forces for implanting (e.g., applying a mallet to a proximal portion of the device 100), etc. In addition, methods of utilizing the device 100 can include coupling the device 100 to an implant at a location remote from a surgical site, e.g., a surgical technician or other user could couple the device to an implant at a “back table” or other staging area, then pass the assembled implant and device to a surgeon or other user to position the implant at a surgical site. Still further, coupling and de-coupling the device 100 to an implant can be done without application of counter-torque forces to the implant because the post 141 prevents relative rotation between the implant and the housing 140 of the device 100 during application of torque to the setscrew 130.

Any of the features described herein in connection with FIGS. 6A-6F can be similarly applied to the embodiment shown in FIGS. 7A-7D. The various figures are provided to illustrate use of the device 100 in connection with different types of implants, such as the connector 10 and pedicle hook 20. The device as described herein, and including any combination of the variously disclosed features, can be utilized with either of these implants or any other implant having features complementary to the device to facilitate coupling thereto.

Various devices and methods disclosed herein can be used in minimally invasive surgery and/or open surgery. While various devices and methods disclosed herein are generally described in the context of surgery on a human patient, the methods and devices disclosed herein can be used in any of a variety of surgical procedures with any human or animal subject, or in non-surgical procedures.

Various devices disclosed herein can be constructed from any of a variety of known materials. Example materials include those that are suitable for use in surgical applications, including metals such as stainless steel, titanium, titanium nitride, nickel, cobalt, chrome, cobalt-chromium, or alloys and combinations thereof, polymers such as PEEK, ceramics, carbon fiber, and so forth. The various components of the devices disclosed herein can be rigid or flexible. In addition, one or more of the components or devices disclosed herein can be formed as monolithic or unitary structures, e.g., formed from a single continuous material, or can be formed from separate components coupled together in a variety of manners that either facilitate or discourage subsequent separation. One or more components or portions of the device can be formed from a radiopaque material to facilitate visualization under fluoroscopy and other imaging techniques, or from a radiolucent material so as not to interfere with visualization of other structures. Example radiolucent materials include carbon fiber and high-strength polymers. Further, various methods of manufacturing can be utilized, including 3D printing or other additive manufacturing techniques, as well as more conventional manufacturing techniques, including molding, stamping, casting, machining, etc.

Various devices or components disclosed herein can be designed to be disposed of after a single use, or they can be designed to be used multiple times. In either case, however, various devices or components can be reconditioned for reuse after at least one use. Reconditioning can include any combination of the steps of disassembly, followed by cleaning or replacement of particular pieces, and subsequent reassembly. In particular, a device or component can be disassembled, and any number of the particular pieces or parts thereof can be selectively replaced or removed in any combination. Upon cleaning and/or replacement of particular parts, the device or component can be reassembled for subsequent use either at a reconditioning facility, or by a surgical team immediately prior to a surgical procedure. Reconditioning of a device or component can utilize a variety of techniques for disassembly, cleaning/replacement, and reassembly. Use of such techniques, and the resulting reconditioned device or component, are within the scope of the present disclosure.

Various devices or components described herein can be processed before use in a surgical procedure. For example, a new or used device or component can be obtained and, if necessary, cleaned. The device or component can be sterilized. In one sterilization technique, the device or component can be placed in a closed and sealed container, such as a plastic or TYVEK bag. The container and its contents can be placed in a field of radiation that can penetrate the container, such as gamma radiation, x-rays, or high-energy electrons. The radiation can kill bacteria on the device or component and in the container. The sterilized device or component can be stored in the sterile container. The sealed container can keep the device or component sterile until it is opened in the medical facility. Other forms of sterilization are also possible, including beta or other forms of radiation, ethylene oxide, steam, or a liquid bath (e.g., cold soak). Certain forms of sterilization may be better suited to use with different devices or components, or portions thereof, due to the materials utilized, the presence of electrical components, etc.

In this disclosure, articles “a” and “an” are used herein to refer to one or to more than one (i.e., at least one) of the grammatical object of the article. By way of example, “an element” means at least one element and can include more than one element. The term “about” is used to provide flexibility to a numerical range endpoint by providing that a given value may be “slightly above” or “slightly below” the endpoint without affecting the desired result. The use herein of the terms “including,” “comprising,” or “having,” and variations thereof, is meant to encompass the elements listed thereafter and equivalents thereof, as well as additional elements. As used herein, “and/or” refers to and encompasses any and all possible combinations of one or more of the associated listed items, as well as the lack of combinations where interpreted in the alternative (“or”). Further, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B,” “one or more of A and B,” and “A and/or B” are each intended to mean “A alone, B alone, or A and B together.” A similar interpretation is also intended for lists including three or more items. For example, the phrases “at least one of A, B, and C,” “one or more of A, B, and C,” and “A, B, and/or C” are each intended to mean “A alone, B alone, C alone, A and B together, A and C together, B and C together, or A and B and C together.” In addition, use of the term “based on,” is intended to mean, “based at least in part on,” such that an un-recited feature or element is also permissible.

To the extent that linear, circular, or other dimensions are used in the description of the disclosed devices and methods, such dimensions are not intended to limit the types of shapes that can be used in conjunction with such devices and methods. Equivalents to such dimensions can be determined for different geometric shapes, etc. Further, like-numbered components of the embodiments can generally have similar features. Still further, sizes and shapes of the devices, and the components thereof, can depend at least on the anatomy of the subject in which the devices will be used, the size and shape of objects with which the devices will be used, and the methods and procedures in which the devices will be used.

The figures provided herein are not necessarily to scale. Still further, to the extent arrows are used to describe a direction of movement, these arrows are illustrative and in no way limit the direction that the respective component can or should be moved. Other movements and directions may be possible to create the desired result in view of the present disclosure.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Further features and advantages based on the above-described embodiments are possible and within the scope of the present disclosure. Accordingly, the disclosure is not to be limited by what has been particularly shown and described. All publications and references cited herein are expressly incorporated herein by reference in their entirety, except for any definitions, subject matter disclaimers, or disavowals, and except to the extent that the incorporated material is inconsistent with the express disclosure herein, in which case the language in this disclosure controls.

Examples of the above-described embodiments can include the following:
1. A surgical instrument, comprising:
   a proximal handle;
   a shaft extending distally from the proximal handle;
   a housing coupled to the distal end of the shaft, the housing including an upper socket, two opposed arms extending from the upper socket, and a post connecting the two opposed arms at a position farthest from the upper socket;
   a setscrew disposed within the housing;
   wherein the two opposed arms of the housing define a gap between the post and the upper socket, and the upper socket includes an opening at an end opposite the two opposed arms; and
   wherein the setscrew is captured within the upper socket and free to translate from a first position, wherein a threaded distal end of the setscrew is disposed within the upper socket, to a second position, wherein the threaded distal end of the setscrew extends into the gap between the two opposed arms.
2. The instrument of example 1, wherein the setscrew is monolithic.
3. The instrument of examples 1 or 2, wherein the housing is monolithic.
4. The instrument of any of examples 1 to 3, wherein the setscrew comprises a proximal end defining a radially outwardly extending flange that is configured to interface with a corresponding shoulder formed in the opening of the upper socket to define a maximum depth of distal insertion of the setscrew.
5. The instrument of example 4, wherein the housing includes a through-hole formed in a sidewall of the upper socket, the instrument further comprising a pin disposed in the through-hole such that the pin extends into the upper socket to capture the setscrew.
6. The instrument of example 5, wherein the housing includes first and second through-holes opposed from one another, the instrument further comprising corresponding first and second pins disposed in the first and second through-holes.
7. The instrument of example 6, wherein a distance between radially inward most ends of the first and second pins is less than an outer diameter of the threaded distal end of the setscrew.
8. The instrument of example 6, wherein the setscrew defines an intermediate portion arranged longitudinally between the threaded distal end and the proximal end of the setscrew, the intermediate portion having a diameter less than a diameter of the threaded distal end and less than the distance between the radially-inward-most ends of the first and second pins, such that the first and second pins permit translation of the setscrew along a length of the intermediate portion and prevent proximal removal of the setscrew by interfering with the threaded distal end.
9. The instrument of any of examples 1 to 8, wherein the housing is coupled with the shaft at one end of the post.
10. The instrument of any of examples 1 to 9, wherein a distal-facing surface of the post is convex.
11. The instrument of any of examples 1 to 10, wherein the two opposed arms define a pair of inner sidewalls that are coextensive with an inner surface of the upper socket.
12. The instrument of any of examples 1 to 11, wherein the threaded distal end of the setscrew is sized and shaped to extend laterally beyond the two opposed arms when the threaded distal end extends into the gap between the arms.
13. The instrument of any of examples 1 to 12, wherein a proximal-facing surface of the setscrew is configured to be flush with a proximal end of the upper socket when the setscrew is in the second position.
14. The instrument of any of examples 1 to 13, wherein a longitudinal axis of the post and a longitudinal axis of the shaft are coplanar.
15. The instrument of any of examples 1 to 13, wherein a longitudinal axis of the post and a longitudinal axis of the shaft are obliquely angled relative to one another in a plane that is perpendicular to a longitudinal axis of the upper socket.
16. The instrument of any of examples 1 to 15, wherein the setscrew is biased toward the opening of the upper socket opposite the two opposed arms.
17. A surgical instrument, comprising:
   a proximal handle;
   a shaft extending distally from the proximal handle;
   a housing coupled to the distal end of the shaft, the housing including an upper socket with a through-hole formed in a sidewall thereof, an arm extending from the upper socket, and a post extending from the arm at a position farthest from the upper socket;
   a setscrew disposed within the housing; and
   a pin disposed in the through-hole such that the pin extends into the upper socket to capture the setscrew.
18. The instrument of example 17, wherein the upper socket includes first and second through-holes opposed from one another, the instrument further comprising corresponding first and second pins disposed in the first and second through-holes.
19. The instrument of example 18, wherein a distance between radially inward most ends of the first and second pins is less than an outer diameter of a threaded distal end of the setscrew.
20. The instrument of example 19, wherein the setscrew defines an intermediate portion arranged longitudinally between the threaded distal end and a proximal end of the setscrew, the intermediate portion having a diameter less than a diameter of the threaded distal end and less than the distance between the radially-inward-most ends of the first and second pins, such that the first and second pins permit translation of the setscrew along a length of the intermediate portion and prevent proximal removal of the setscrew by interfering with the threaded distal end.
21. The instrument of any of examples 17 to 20, wherein the setscrew comprises a proximal end defining a radially outwardly extending flange that is configured to interface with a corresponding shoulder formed in an opening of the upper socket at an end opposite the arm to define a maximum depth of distal insertion of the setscrew.
22. The instrument of any of examples 17 to 21, wherein the setscrew is biased toward an end of the upper socket that is opposite the arm extending from the upper socket.
23 A method of assembling an instrument, comprising:
   inserting a setscrew into a proximal-facing opening of an upper socket of a housing, the setscrew including a threaded distal end, a proximal end defining a radially-outward extending flange, and an intermediate portion extending therebetween that has a diameter less than the threaded distal end and the radially-outward extending flange;
   inserting first and second pins into opposed first and second through-holes formed in a sidewall of the upper socket such that a distance between radially-inward-most ends of the first and second pins is less than an outer diameter of the threaded distal end of the setscrew, less than a diameter of the radially-outward extending flange of the setscrew, and greater than a diameter of the intermediate portion of the setscrew such that the first and second pins capture the setscrew within the upper socket while allowing translation of the setscrew relative to the upper socket along a length of the intermediate portion of the setscrew.
24. The method of example 23, further comprising coupling a shaft to the housing.
25. The method of example 24, further comprising coupling a handle to the shaft.
26 The method of any of examples 23 to 25, further comprising extending a post of the housing that is positioned distal to the upper socket into a recess of an implant.
27. The method of example 26, further comprising distally extending the setscrew relative to the upper socket such that the threaded distal interfaces with threads formed in the recess of the implant.
28. The method of example 27, further comprising rotating the setscrew relative to the upper socket to urge a distal-facing surface of the upper socket into contact with a proximal-facing surface of the implant and create a rigid coupling between the housing and the implant.

1. A surgical instrument, comprising:
a proximal handle;
a shaft extending distally from the proximal handle;
a housing coupled to a distal end of the shaft, the housing including an upper socket, two opposed arms extending from the upper socket, and a post connecting the two opposed arms at a position farthest from the upper socket;
a setscrew disposed within the housing;
wherein the two opposed arms of the housing define a gap between the post and the upper socket, and the upper socket includes an opening at an end opposite the two opposed arms; and
wherein the setscrew is captured within the upper socket and free to translate from a first position, wherein a threaded distal end of the setscrew is disposed within the upper socket, to a second position, wherein the threaded distal end of the setscrew extends into the gap between the two opposed arms.

2. The instrument of claim 1, wherein the setscrew is monolithic.

3. The instrument of claim 1, wherein the housing is monolithic.

4. The instrument of claim 1, wherein the setscrew comprises a proximal end defining a radially outwardly extending flange that is configured to interface with a corresponding shoulder formed in the opening of the upper socket to define a maximum depth of distal insertion of the setscrew.

5. The instrument of claim 4, wherein the housing includes a through-hole formed in a sidewall of the upper socket, the instrument further comprising a pin disposed in the through-hole such that the pin extends into the upper socket to capture the setscrew.

6. The instrument of claim 5, wherein the housing includes first and second through-holes opposed from one another, the instrument further comprising corresponding first and second pins disposed in the first and second through-holes.

7. The instrument of claim 6, wherein a distance between radially inward most ends of the first and second pins is less than an outer diameter of the threaded distal end of the setscrew.

8. The instrument of claim 7, wherein the setscrew defines an intermediate portion arranged longitudinally between the threaded distal end and the proximal end of the setscrew, the intermediate portion having a diameter less than a diameter of the threaded distal end and less than the distance between the radially-inward-most ends of the first and second pins, such that the first and second pins permit translation of the setscrew along a length of the intermediate portion and prevent proximal removal of the setscrew by interfering with the threaded distal end.

9. The instrument of claim 1, wherein the housing is coupled with the shaft at one end of the post.

10. The instrument of claim 1, wherein a distal-facing surface of the post is convex.

11. The instrument of claim 1, wherein the two opposed arms define a pair of inner sidewalls that are coextensive with an inner surface of the upper socket.

12. The instrument of claim 1, wherein the threaded distal end of the setscrew is sized and shaped to extend laterally beyond the two opposed arms when the threaded distal end extends into the gap between the arms.

13. The instrument of claim 1, wherein a proximal-facing surface of the setscrew is configured to be flush with a proximal end of the upper socket when the setscrew is in the second position.

14. The instrument of claim 1, wherein a longitudinal axis of the post and a longitudinal axis of the shaft are coplanar.

15. The instrument of claim 1, wherein a longitudinal axis of the post and a longitudinal axis of the shaft are obliquely angled relative to one another in a plane that is perpendicular to a longitudinal axis of the upper socket.

16. The instrument of claim 1, wherein the setscrew is biased toward the opening of the upper socket opposite the two opposed arms.

17. A surgical instrument, comprising:
a proximal handle;
a shaft extending distally from the proximal handle;
a housing coupled to a distal end of the shaft, the housing including an upper socket with a through-hole formed in a sidewall thereof, an arm extending from the upper socket, and a post extending from the arm at a position farthest from the upper socket;
a setscrew disposed within the housing; and
a pin disposed in the through-hole such that the pin extends into the upper socket to capture the setscrew.

18. The instrument of claim 17, wherein the upper socket includes first and second through-holes opposed from one another, the instrument further comprising corresponding first and second pins disposed in the first and second through-holes.

19. The instrument of claim 18, wherein a distance between radially inward most ends of the first and second pins is less than an outer diameter of a threaded distal end of the setscrew.

20. The instrument of claim 19, wherein the setscrew defines an intermediate portion arranged longitudinally between the threaded distal end and a proximal end of the setscrew, the intermediate portion having a diameter less than a diameter of the threaded distal end and less than the distance between the radially-inward-most ends of the first and second pins, such that the first and second pins permit translation of the setscrew along a length of the intermediate portion and prevent proximal removal of the setscrew by interfering with the threaded distal end.

21. The instrument of claim 17, wherein the setscrew comprises a proximal end defining a radially outwardly extending flange that is configured to interface with a corresponding shoulder formed in an opening of the upper socket at an end opposite the arm to define a maximum depth of distal insertion of the setscrew.

22. The instrument of claim 17, wherein the setscrew is biased toward an end of the upper socket that is opposite the arm extending from the upper socket.

23-28. (canceled)
